(19) United States
(12) Patent Application Publication
(10) Pub. No.: US 2024/0058228 A1
(43) Pub. Date: Feb. 22, 2024

(54) DRY SHAMPOO PRODUCT WITH 55% OR LESS VOLATILE ORGANIC COMPOUNDS

(71) Applicant: The Procter & Gamble Company, Cincinnati, OH (US)

(72) Inventors: Jodi Lee Brown, Cincinnati, OH (US); Casey Robert Albertz, Miami Heights Township, OH (US)

(21) Appl. No.: 17/892,194

(22) Filed: Aug. 22, 2022

(51) Int. Cl. A61K 8/04; A61K 8/58; A61Q 5/00

(52) U.S. Cl. CPC A61K 8/046; A61K 8/585; A61Q 5/008; A61K 2800/30; A61K 2800/87

(57) ABSTRACT

A dry shampoo product that can contain a carrier comprising a volatile silicone having a viscosity of 1 cst or less, an oil absorbing starch dispersed in the volatile silicone carrier, and a propellant. The dry shampoo product can have 55% or less of volatile organic compounds, by weight of the dry shampoo product, while also delivering acceptable product performance.

### FIELD OF THE INVENTION

A dry shampoo product that includes an oil absorbing starch dispersed in a carrier and a propellant, particularly an aerosol dry shampoo product with 55% or less violative organic compounds.

### BACKGROUND OF THE INVENTION

Many consumers use dry shampoo, which does not require the use of water, to reduce oil and dirt in their hair and on their scalp between washes. Consumers want a dry shampoo that is fast drying, easy to apply, and makes their hair look and feel less greasy and residue free.

Typical dry shampoo products include ethanol as the carrier (a volatile organic compound or VOC), one or more sebum absorbing powders, and a hydrocarbon propellant (also a VOC) and therefore, these products typically have between 80-90% VOC content. However, there is both regulatory and consumer pressure to lower the concentration of VOCs in dry shampoo products to 55% or less, and preferably 50% or less.

Therefore, there is a need for a dry shampoo product that has 50% or less VOC and dries quickly, effectively reduces oil on a user's hair and scalp, and does not leave behind a noticeable residue.

### SUMMARY OF THE INVENTION

A dry shampoo product, comprising: (a) a carrier comprising a volatile silicone having a viscosity of 1 cst or less; (b) an oil absorbing starch dispersed in the volatile silicone carrier; (c) a propellant; wherein the dry shampoo product comprises 55% or less of volatile organic compounds, by weight of the dry shampoo product.

An aerosol hair care product, comprising: (a) an aerosol container comprising a pressurizable container and an actuator; and (b) a dry shampoo product disposed in the pressurizable container, the dry shampoo product, comprising: (i) a dry shampoo composition comprising: (1) a carrier comprising hexamethyldisiloxane having a viscosity of 0.65 cst or less; (2) an oil absorbing starch dispersed in the hexamethyldisiloxane volatile silicone carrier; (ii) a propellant; wherein the dry shampoo product comprises 55% or less of volatile organic compounds, by weight of the dry shampoo product; wherein the aerosol personal care product is pressurized to greater than about 50 psig.

### DETAILED DESCRIPTION OF THE INVENTION

Dry shampoo products are spray hair products that reduce the appearance of oil and dirt on a user's hair and/or scalp. Unlike traditional shampoos and conditioners, dry shampoo can be applied to dry hair and does not need to be washed out. It is frequently used to refresh hair between washes.

Typical dry shampoo products contain between 80-90% VOC content. Ethanol (a VOC) is generally used as the bulk phase and it helps suspend oil-absorbing particles and deliver them to the user's hair and scalp and draws moisture and oil away from a user's hair and scalp, while also drying quickly. The propellant is generally a hydrocarbon (another VOC) that acts as a solvent inside the aerosol package and when the actuator button is pressed, a valve opens and since the pressure outside the can is less than the pressure inside, the propellant expands, pushing the product up a dip tube and out through the valve in a fine mist.

There is both regulatory and consumer pressure to lower the concentration of VOCs in dry shampoo products to 55% or less, and preferably 50% or less. This requires reformulation to remove some or all of the ethanol carrier and/or the hydrocarbon VOC propellant.

Some products may replace hydrocarbon propellants with HFC-152a propellant (1,1 Difluoroethane), which is a liquified gas propellant that is not a VOC and is known to have similar propellant properties to hydrocarbon VOC propellants. However, there are also pressures from some regulatory agency and consumers to avoid using this propellant and therefore this is not a preferred solution for the dry shampoo products described herein.

Removing and replacing the ethanol is challenging, since ethanol is key to providing important performance attributes for dry shampoo formulations. These performance attributes include the following:
Quick evaporation that leads to a fast dry time/no wet hair feel
Ability to dissolve scalp oil for clean look dry shampoo benefit
Odorless
Low viscosity
Low surface tension
Compatible with most surfaces

It was found that using hexamethyldisiloxane (HMDS), the lowest molecular weight linear volatile silicone, with a viscosity of 1 cSt or less, preferably 0.65 cSt or less, as the carrier in a dry shampoo product can create a product with consumer acceptable performance that also has 55% or less VOCs, preferably 50% or less VOCs. The dry shampoo products with an HMDS carrier can deliver conditioning and/or smoothing and/or frizz reducing benefits to a user's hair. Another benefit of HMDS, is that the spray feels like it is room temperature when applied to the hair, while traditional ethanol-based dry shampoos feel cool when they are dispensed. The viscosity can be determined according to the Viscosity Test Method, described herein.

Interestingly, HMDS is sometimes used as a minor ingredient at low levels (i.e., much less than 20%) in hair care formulations, including ethanol and water-based hair styling products. However, it was unexpected that HMDS, a silicone material, which some users think makes their hair feel heavy and greasy, would perform well as the carrier of a dry shampoo aerosol spray. The main benefit of a dry shampoo product is to absorb hair oil and reduce the appearance of oily roots, therefore adding a silicone oil carrier for this purpose is not intuitive.

The dry shampoo product (i.e., the dry shampoo composition and the propellant) can include ≥20% HMDS, alternatively ≥25%, alternatively ≥30%, alternatively ≥35%, and alternatively ≥40%. The dry shampoo product can include from about 20% to about 60% HMDS, alternatively from about 25% to about 55%, alternatively from about 30% to about 50%, alternatively from about 35% to about 45%, and alternatively from about 38% to about 43%.

The dry shampoo compositions can be formulated without, substantially free of, or free of ethanol, water, surfactant, parabens, propellant 152a (1, 1 Difluoroethane), and/or polymers including, but not limited to, film-forming polymers.

The ejected composition can include particles having an average particle size distribution (Dv50) from about 5 μm to about 250 μm and alternatively from about 10 μm to about 225 μm. The average particle size distribution (Dv50) is determined by the Particle Size Distribution Test Method, described hereafter. Such average particle size distribution is useful for providing optimal drying time on hair since the particles of the ejected composition are well spread across the head of hair and yet there is enough oil absorbing starch in each particle (droplet) to provide sufficient oil absorption on the hair and scalp.

The spray rate of the ejected composition can be from about 0.4 g/s to about 0.9 g/s, alternatively from about 0.45 g/s to about 0.75 g/s, alternatively from about 0.5 g/s to about 0.7 g/s, alternatively from about 0.55 g/s to about 0.7 g/s, and alternatively from about 0.57 g/s to about 0.65 g./s. The spray rate can be determined by the Delivery Rate Test Method, described herein. The ejected composition can deliver from about 0.025 g/s to about 0.2 g/s of oil absorbing starch, alternatively from about 0.05 g/s to about 0.15 g/s, alternatively from about 0.06 g/s to about 0.1 g/s, alternatively from about 0.07 g/s to about 0.08 g/s, and alternatively about 0.075 g/s. The amount of oil absorbing starch can be determined according to the Delivery Rate Test Method, described herein.

The pressure in the aerosol container can be greater than 50 psig. The pressure in the aerosol container can be from about 40 psig to about 85 psig, alternatively from about 45 psig to about 80 psig, alternatively from about 50 psig to about 75 psig, and alternatively from about 55 psig to about 70 psig, according to the Pressure Test Method, described herein.

The dry shampoo composition can have a viscosity ≥800 cP, alternatively ≥1250 cP, alternatively ≥3000 cP, and alternatively ≥5000 cP. The dry shampoo composition can have a viscosity from about 1000 cP to about 12,000 cP. The viscosity can be determined by the Viscosity Test Method, described herein.

The dry shampoo composition can feel dry within 10 seconds of being applied to the hair, alternatively within 7 seconds, and alternatively within 5 seconds.

In some examples, the dry shampoo product and/or dry shampoo composition can have a limited number of ingredients, in particular a limited number of ingredients that need to be reported on the INCI (International Nomenclature Cosmetic Ingredient) statement on the package. The composition can ≤25 ingredients, alternatively ≤20 ingredients, alternatively ≤15 ingredients, alternatively ≤12 ingredients, alternatively ≤10 ingredients, and alternatively ≤8 ingredients.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

### Definitions

As used herein, “formulated without” means that the ingredient is not intentionally added. However, “formulated without” does not guarantee “100% free from” since trace contaminants are possible.

As used herein, “hair” means mammalian hair including scalp hair, facial hair and body hair, particularly on hair on the human head and scalp.

As used herein, “volatile” means those materials that are liquid under ambient conditions and which have a measurable vapor pressure at 25° C. Specifically the material have a boiling point at 1 atm of about 260° C. or less, preferably about 200° C. or less, more preferably about 150° C. or less and most preferably 100° C. or less.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

### Dry Shampoo Composition

The dry shampoo composition can include an HMDS carrier and an oil absorbing starch. The dry shampoo composition can also contain optional ingredients including rheology modifiers, polar activators, fragrances, and other minor ingredients.

The dry shampoo composition can have a pH of from about 3 to about 9, and alternatively from about 4 to about 6.

### Oil Absorbing Starch

The dry shampoo product (i.e., the dry shampoo composition and the propellant) may include from about 1% to about 15% oil absorbing starch, alternatively from about 3% to about 12%, and alternatively from about 6% to about 10%, by weight of the dry shampoo product. The oil absorbing starch can be dispersed in the carrier.

Non-limiting examples of oil absorbing starch suitable for dry shampoo can include rice starch, corn starch (also known as maize starch), potato starch, silica, clays, and/or tapioca starch. Additional starches that can be suitable for this invention can be found in U.S. Pub. No. 2016/0317396, incorporated by reference.

In some examples, the oil absorbing starch can be a particulate tapioca starch. The particulate tapioca starch may be selected from the group consisting of hydrophobically modified particulate tapioca starch, hydrophobically unmodified particulate tapioca starch, and combinations thereof. The ratio of hydrophobic ally modified particulate tapioca starch to unmodified particulate tapioca starch may be 2:1 or greater.

Hydrophobically modified particulate tapioca starches may be made by a variety of methods, including those discussed in U.S. Pat. Nos. 7,375,214, 7,799,909, 6,037,466, 2,852,404, 5,672,699, and 5,776,476.

Modified tapioca particulate starch may be an organically modified particulate tapioca starch or a silicone grafted particulate tapioca starch. Silicone grafted particulate tapioca starch may be purchased under the trade name Dry Flo TS and under the INCI name Tapioca Starch Polymethylsilsesquioxane. Silicone modified particulate tapioca starch may be produced by a reaction of methyl sodium siliconate (polymethylsilsesquioxane) and tapioca starch. Particulate tapioca starch may be sourced from the Cassava root by standard means know in the art. One example of a commercially available silicone modified particulate tapioca starch is CAS no. 68989-12-8.

### Rheology Modifier

In some examples, the dry shampoo composition can include one or more rheology modifiers. The dry shampoo product (i.e., the dry shampoo composition and the propellant) can include from about 0.5% to about 3% rheology modifier, alternatively from about 0.75% to about 2%, alternatively from about 1% to 1.75%, and alternatively from about 1.25% to about 1.5%.

The dry shampoo composition can include a rheology modifier that may be a water-swellable clays non-limiting examples include laponite, bentolite, montmorilonite, smectite, and hectorite.

The rheology modifiers can be an alginic acid-based materials; non-limiting examples can include sodium alginate, and alginic acid propylene glycol esters.

The rheology modifier may be a homopolymers based on acrylic acid, methacrylic acid or other related derivatives, non-limiting examples can include polyacrylate, polymethacrylate, polyethylacrylate, and polyacrylamide.

The rheology modifiers may be a crosslinked acrylic polymers, a non-limiting example can include carbomers.

The rheology modifier may be an associative polymeric thickeners, non-limiting examples can include: Hydrophobically modified cellulose derivatives; Hydrophobically modified alkoxylated urethane polymers, nonlimiting example include PEG-150/decyl alcohol/SMDI copolymer, PEG-150/stearyl alcohol/SMDI copolymer, polyurethane-39; Hydrophobically modified, alkali swellable emulsions, non-limiting examples include hydrophobically modified polyacrylates, hydrophobically modified polyacrylic acids, and hydrophobically modified polyacrylamides; hydrophobically modified polyethers wherein these materials may have a hydrophobe that can be selected from cetyl, stearyl, oleayl, and combinations thereof, and a hydrophilic portion of repeating ethylene oxide groups with repeat units from 10-300, in another embodiment from 30-200, in a further embodiment from 40-150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

The rheology modifier may be cellulose and derivatives; nonlimiting examples can include microcrystalline cellulose, carboxymethylcelluloses, hydroxyethylcellulose, hydroxypropylcellulose, hydroxypropylmethylcellulose, methylcellulose, ethylcellulose, nitro cellulose, cellulose sulfate, cellulose powder, and hydrophobically modified celluloses.

The rheology modifier may be a guar and guar derivatives; nonlimiting examples include hydroxypropyl guar, and hydroxypropyl guar hydroxypropyl trimonium chloride.

The rheology modifier may be, dibenzylidene sorbitol, karaggenan, pectin, agar, quince seed (Cydonia oblonga Mill), starch-derivatives (e.g. carboxymethyl starch, methylhydroxypropyl starch), algae extracts, dextran, succinoglucan, and pulleran.

The rheology modifier may be polyethylene oxide, polypropylene oxide, and POE-PPO copolymers.

The rheology modifier may be polyvinylpyrrolidone, crosslinked polyvinylpyrrolidone and derivatives. In a further embodiment, the rheology modifier may be polyvinyalcohol and derivatives.

The rheology modifier may be polyethyleneimine and derivatives.

The rheology modifier may be silicas; nonlimiting examples include fumed silica, precipitated silica, and silicone-surface treated silica.

Additional rheology modifiers that may be used are disclosed in U.S. Pat. No. 11,129,780, incorporated by reference.

### Polar Activators

In some examples, the dry shampoo composition can include one or more polar activators. The dry shampoo product (i.e., the dry shampoo composition and the propellant) can include from about 0.1% to about 1% polar activators, alternatively from about 0.2% to about 0.8%, alternatively from about 0.3% to 0.7%, and alternatively from about 0.4% to about 0.6%.

Non-limiting examples of polar activators can include methanol, ethanol, acetone, propylene carbonate, water, triethyl citrate, propylene carbonate, or a combination thereof.

The dry shampoo product can have better homogeneity of the oil absorbing starch suspended in the carrier compared to a dry shampoo product that does not include the rheology modifier when a rheology modifier and/or a polar activator is included in the formulation.

### Optional Ingredients

The dry shampoo compositions described herein may also comprise any other suitable optional ingredients as desired. For example, the hair care compositions may comprise other active or inactive ingredients.

The dry shampoo composition can include a natural oil such as olive oil, argan oil, jojoba oil, passion fruit oil.

The dry shampoo composition can include a scalp health active to provide scalp benefits. This group of materials is varied and provides a wide range of benefits including anti-dandruff, anti-fungal, anti-microbial, moisturization, barrier improvement, and anti-oxidant, anti-itch, and sensates. Such health actives include but are not limited to: zinc pyrithione, climbazole, octopirox, vitamin E and F, salicylic acid, glycols, glycolic acid, PCA, PEGs, erythritol, glycerin, lactates, hyaluronates, allantoin and other ureas, betaines, sorbitol, glutamates, xylitols, menthol, menthyl lactate, isocyclomone, benzyl alcohol, and natural extracts/oils including peppermint, spearmint, argan, jojoba and aloe. The compositions may include other common hair ingredients.

The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: aesthetic components such as fragrances, pigments, colorings/colorants, essential oils, sensates, anti-foaming agents, antimicrobial agents, biological additives, buffering agents, bulking agents, chelating agents, chemical additives, astringents, biocides, film formers or materials, pH adjusters, reducing agents, sequestrants, and surfactants.

### Compressed Propellant

The dry shampoo product (i.e., the dry shampoo composition and the propellant) described herein may comprise from about 30% to about 70%, alternatively from about 40% to about 65%, alternatively from about 49% to about 55%, by weight of the dry shampoo product (i.e. the dry shampoo composition and the propellant).

The propellant may comprise one or more volatile materials, which in a gaseous state, may carry the other components of the dry shampoo composition in particulate or droplet form. The propellant may have a boiling point within the range of from about −45° C. to about 5° C. The propellant may be liquefied when packaged in convention aerosol containers under pressure. The rapid boiling of the propellant upon leaving the aerosol foam dispenser may aid in the atomization of the other components of the dry shampoo product.

Aerosol propellants which may be employed in the dry shampoo product may include the chemically-inert hydrocarbons such as propane, n-butane, isobutane, cyclopropane, and mixtures thereof, as well as halogenated hydrocarbons such as dichlorodifluoromethane, 1,1-dichloro-1,1,2,2-tetrafluoroethane, 1-chloro-1,1-difluoro 2,2-trifluoroethane, 1-chloro-1,1-difluoroethylene, 1,1-difluoroethane, dimethyl ether, monochlorodifluoromethane, trans-1,3,3,3-tetrafluoropropene, and mixtures thereof. The propellant may comprise hydrocarbons such as isobutane, propane, and butane these materials may be used for their low ozone reactivity and may be used as individual components where their vapor pressures at 21.1° C. range from about 1.17 Bar to about 7.45 Bar, alternatively from about 1.17 Bar to about 4.83 Bar, and alternatively from about 2.14 Bar to about 3.79 Bar.

### Method of Using

The dry shampoo product can be used to clean and/or refresh hair in 60 seconds or less. The dry shampoo product can have no noticeable residue and can remove extra oil from any hair type. The dry shampoo product can be sprayed on dry or damp hair. The spray should be held about 8 to about 12 inches from where the spray is intended to land. The dry shampoo can be sprayed on any portion of the hair from the roots to the tips. In some examples, the dry shampoo is sprayed on the roots and massaged into the roots. In other examples, the dry shampoo is sprayed on the hair and combed through. The dry shampoo is not rinsed out.

### Packaging

The aerosol hair care product can include an aerosol dispenser with a pressurizable outer container usable for such a dispenser. The outer container may comprise plastic or metal. The outer container may have an opening. The opening is typically at the top of the pressurizeable container when the pressurizeable container is in its-in use position. The opening defines a neck, to which other components may be sealed.

A valve cup may be sealed to the opening of the outer container. A valve assembly, in turn, may be disposed within the valve cup. The valve assembly provides for retention of the dry shampoo product (dry shampoo composition and propellant) within the aerosol dispenser until the dry shampoo product is selectively dispensed by a user. The valve assembly may be selectively actuated by an actuator. Selective actuation of the valve assembly allows the user to dispense a desired quantity of the dry shampoo composition on demand Additional details and examples regarding aerosol dispensers are found in U.S. Pat. No. 9,505,509, incorporated by reference (see e.g., FIG. 2B showing an aerosol dispenser with a dip tube and accompanying text).

Test Methods

### Delivery Rate Test Method

The delivery rate of the aerosol hairspray product herein is determined following ASTM D 3069-94, “Standard Test Method for Delivery Rate of Aerosol Products.” In this test, the delivery rate of the product is determined by measuring the mass lost in a given time period. This correlates with the quantity of material expelled though the valve and actuator combination in a given time period. In this case, the can is tested at room temperature (at 21° C.) and a duration of 2 sec to 10 sec for the actuation time. The delivery rate (also referred to as the spray rate) is then determined by the equation:

Delivery Rate (g/s)=Mass loss (g)/Actuation time (s)

The delivery rate of the oil absorbing starch is determined by the following equation:

Delivery Rate of Oil Absorbing Starch (g/s)=Delivery rate (g/s)*% Oil absorbing starch in dry shampoo product

### Oil Absorption Test Method

Wash 5 g, 8 inch long, straight hair switch with Pantene® Nutrient Blends Pure Clean & Clarify Silicone-Free Shampoo and hang to let air dry. At least one switch is used for each dry conditioner composition being tested, at least one switch is the negative control, and at least one positive control. The positive control is a clean, untreated hair switch.

Weigh 0.05 g of artificial sebum per gram of hair. While wearing gloves, apply artificial sebum to tips of fingers, using both hands rub tips of fingers together to melt the sebum. Apply sebum to front and back of hanging switch in downward motion, then milk into hair (hand over hand motion) for 10-15 seconds. Place sebum treated hair switch in between two pieces of foil. Blow dry front and back of switch, 30 seconds per side. This will ensure even distribution of sebum through the entirety of the switch. Hang switch.

Create at least one negative control by placing a wide tooth comb through one of the hair switches, making sure the hair is evenly distributed between the comb teeth. Slowly move the comb down the hair swatch one time to create brush lines.

Repeat the steps above for switches to be treated with dry shampoo. Spray dry shampoo from the top to the bottom of the switch over 2-3 seconds, repeat on the back of the switch. Make sure there is 6 inches between the actuator and the hair switch. Supporting the back of the switch with opposite hand to avoid the hair being blown. Then, let the switch sit for 30 seconds and then brush through switch from top to bottom (3 times per side). Next, use a wide tooth comb to create “brush lines,” as described in creating the negative control, above.

Observe the changes in the amount and brush lines and changes in brush line definition and thickness with an unaided eye (excepting standard corrective lenses adapted to compensate for near-sightedness, farsightedness, or stigmatism, or other corrected vision) in lighting at least equal to the illumination of a standard 100-watt incandescent white light bulb at 30 cm. Compare the one or more switches treated with dry shampoo with the negative control and positive control.

### Particle Size Distribution Test Method

A Malvern Spraytec instrument is used to measure the particle size distribution. The Dv50 is the term to describe the maximum particle size diameter below which 50% of the sample volume possesses, also known as the median particle size by volume. The Malvern Spraytec instrument uses the technique of laser diffraction for measurement of the size of the spray particles. The intensity of light scattered as a laser beam passes through a spray is measured. This data is then analysed to calculate the size of the particles that created the scattering pattern. A Malvern Spraytec 2000 is used according to the manufacturer's instructions. Test samples have a temperature between 20° C. to 22° C.

### Pressure Test Method

The pressure test is performed at 20 to 24° C. Shake the aerosol container and then actuate the package briefly (˜1 second), to ensure the full product is in the dip tube and not just the propellant. If an actuator button or actuator spray cap has been placed on the aerosol valve, then first remove the button or spray cap. Measure, and record the can pressure using a calibrated pressure gauge. The pressure read by the gauge can be recorded to the nearest 0.5 psi.

### Viscosity Test Method

The viscosities are measured with a Brookfield DV2T Viscometer on the dry shampoo composition. In some cases, the can is degassed and then the viscosity of the dry shampoo composition is measured. The Brookfield Viscometer determines viscosity by measuring the force to turn the spindle in the solution at a given rate. The spindle used is an RV-4 at a product temperature of 20.5 Celsius a speed setting of 5 rpm and at a measurement time of 45 seconds. A minimum sample size of 250 mL is required.

### Visible Residue Test Method

5 g, 8 inch long, straight hair switch are wetted for 15 seconds and shampooed with Pantene® Nutrient Blends Pure Clean & Clarify Silicone-Free Shampoo (0.1 ml of shampoo per 1 g of hair) and hang to let air dry. The switches are hung and dried overnight.

Once dried, 3 switches were set aside as positive controls and the rest of the switches were treated with sebum, as described in the Oil Absorption Test Method, described herein.

Spray dry shampoo from the top to the bottom of the switch over 2-3 seconds, while supporting the back of the switch with opposite hand to avoid the hair being blown. Make sure there is 6 inches between the actuator and the hair switch. Massage the dry shampoo into the hair using a circular motion with thumbs from the top to the bottom of the switch. Then, repeat on the other side. Once both sides are treated, the switch is brushed through 3 times on the front and 3 times on the back of the switch. Any static is removed from all switches via static gun.

Observe the changes between the control switches and the treated switches. The switches are observed with an unaided eye (excepting standard corrective lenses adapted to compensate for near-sightedness, farsightedness, or stigmatism, or other corrected vision) in lighting at least equal to the illumination of a standard 100-watt incandescent white light bulb at 60 cm. Compare the one or more switches treated with dry shampoo with the controls.

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight of the dry shampoo product (i.e., the dry shampoo composition and the propellant) unless otherwise specified.

A variety of dry shampoo compositions were made containing the contents in Table 1 and Table 2.

Examples 1˜4 in Table 1, below, are dry shampoo products that contain ethanol or HMDS, an oil absorbing starch, minors, fragrance, optionally silica, and propellant. The oil absorbing starch in these examples is modified tapioca starch and/or a combination of modified tapioca starch and unmodified tapioca starch. It is believed that other oil absorbing starches that are used in dry shampoo (e.g., rice starch, corn starch, and/or potato starch) have similar oil absorbing properties. The weight % propellant is determined when the dry shampoo product is in a full aerosol container. The total % VOC is calculated by adding the weight percent of each VOC ingredient (e.g., ethanol and/or propellant). Each example is tested for the following:
Dry time was determined by spraying the hair swatch with dry shampoo, waiting approximately 10 seconds, and then feeling the hair swatch to determine if it feels dry.
Oil absorption at hair roots, according to the Oil Absorption Test Method, described herein
Visible residue after application, determined by the Visible Residue Test Method, described herein
Pressure, according to the Pressure Test Method, described herein.
Average particle size (Dv50), determined by the Particle Size Distribution Test Method, described herein
Spray rate, determined by the Delivery Rate Test Method, described herein

TABLE 1

| | Ex. 1 | Ex. 2 | Ex. 3 | Ex. 4 |
|---|---|---|---|---|
| | (wt. %) | (wt. %) | (wt. %) | (wt. %) |
| Ethanol | 11.31 | 11.28 | — | — |
| Hexamethyldisiloxane | — | — | 41.87 | 42.37 |
| Modified Tapioca | 8.51 | 8.51 | 8.51 | 6.5 |
| Starch | | | | |
| Tapioca Starch | — | — | — | 1.2 |
| Silica | — | — | — | 0.25 |
| Minors | 0.06 | 0.06 | 0.06 | 0.06 |
| Fragrance | 0.12 | 0.12 | 0.12 | 0.12 |
| Dimethyl ether | 80 | 38.4 | 49.50 | 49.50 |
| HFC-152a | — | 41.6 | — | — |
| Total % VOC | 91.31% | 49.68% | 49.50% | 49.50% |
| Dry Time | Control | Approximately | Approximately | Approximately |
| | | Equal to | Equal to | Equal to |
| | | Example 1 | Example 1 | Example 1 |
| Oil absorption at hair | Control | Approximately | Approximately | Approximately |
| roots | | Equal to | Equal to | Equal to |
| | | Example 1 | Example 1 | Example 1 |
| Visible white powder | Control | Approximately | Less than | Less than |
| residue | | Equal to | Example 1 | Example 1 |
| | | Example 1 | | |
| Spray Properties | 50 | 61 | 57 | 57 |
| Pressure, psig | | | | |
| Average particle size, | 13.72 | 13.60 | 17.61 | 18.86 |
| Dv50, μm | | | | |
| Spray Rate, g/s | 0.60 | 0.68 | 0.59 | 0.62 |

In Table 1, Example 1 is a current dry shampoo product with a hydrocarbon propellant and ethanol, which are both VOCs, and modified tapioca starch. Example 1 is known to have consumer preferred dry time, oil absorption at hair roots, visible residue after application, and spray properties that include pressure, average particle size (Dv50), and spray rate.

Example 2 is a dry shampoo product that is like Example 1, except a portion of the hydrocarbon propellant is replaced with HFC-152a (1,1-Difluoroethane), which is an organoflurine (i.e., not a VOC). It was found that the dry time, oil absorption, and visible residue were approximately equal to those of Example 1. The spray properties that include pressure, average particle size, and spray rate are close to those measured for Example 1 and it is believed that the small variation would not be noticeable to consumers. Example 2 has a total VOC content of 49.68%. However, HFC-152a may not be consumer and industry preferred.

Examples 3 and 4 are like Example 1, except the ethanol is removed and HMDS is added. Therefore, the total % VOC in the compositions is 49.5%. In addition, Examples 3 and 4 are both ethanol and water free. The oil absorbing starch in Example 3 is modified tapioca starch, like Examples 1 and 2. The oil absorbing starch in Example 4 is a combination of modified tapioca starch and tapioca starch. It was found that the dry time and oil absorption for Examples 3 and 4 were like Example 1. There was less visible white powder in Examples 3 and 4 than in Example 1, which could be preferable to consumers. In one experiment, hair switches were treated with 0.005 g of sebum/gram of hair and washed with a non-conditioning shampoo (Pantene® Nutrient Blends Pure Clean & Clarify Silicone-Free Shampoo) and the other was treated with Example 3 and Example 3 had the same residue as the non-conditioning shampoo, which is generally an excellent cleaning shampoo that leaves little residue. The spray properties that include pressure, average particle size, and spray rate are close to those measured for Example 1 and it is believed that the small variation would not be noticeable to consumers.

The results in Table 1, show that Examples 3 and 4, which are ethanol and water free, contain HMDS and have a total VOC % less than or equal to 50% have similar spray properties and performance to Example 1, which includes ethanol and has a total VOC content greater than 90%. It is believed that consumers will like the performance of Examples 3 and 4 just as much, if not more, than they like the performance of Example 1.

Table 2, shows an example of a dry shampoo composition that was made that contains HMDS and hectorite clay. It was found that in large batch formulas that contained oil absorbing starch, in particular modified tapioca starch, and HMDS, the tapioca starch would settle before the aerosol containers were filled. It was that hectorite clay could be added to the composition, which can help to keep the starch suspended and can also act as an anti-caking agent. If too much clay was added, it was difficult to process the dry shampoo composition. However, if too little clay was added, then the starch was not suspended, and it settled out. It was also found that adding triethyl citrate with the hectorite clay can help promote the clay network and prevent agglomeration and settling.

TABLE 2

| | Ex. A (wt. %) |
|---|---|
| Hexamethlydisiloxane | 39.85% |
| Disteardimonium Hectorite | 1.505% |
| Triethyl Citrate | 0.505% |
| Modified Tapioca Starch - Tapioca | |
| Starch (and) | 8.51% |
| Polymethylsilsesquioxane² | |
| Fragrance | 0.12% |
| Dimethyl Ether | 49.5% |

Tradenames and Suppliers for Table 2
1. Bentone ® 38 from Elementis ® Global
²Dry-Flo ® TS Pure from Akzo Nobel ®

### Combinations

A. A dry shampoo product, comprising:
a) a carrier comprising a volatile silicone having a viscosity of 1 cst or less, preferably 0.65 cst or less;
b) an oil absorbing starch dispersed in the volatile silicone carrier;
c) a propellant;
   wherein the dry shampoo product comprises 55% or less of volatile organic compounds, by weight of the dry shampoo product, preferably 50% or less volatile organic compounds.
      B. The dry shampoo product according to Paragraph A, wherein the volatile silicone is hexamethyldisiloxane.
      C. The dry shampoo product according to Paragraph B, wherein the hexamethyldisiloxane is present at greater than 20%, by weight of the dry shampoo product, preferably ≥30%, more preferably ≥35%, and even more preferably ≥40%.
      D. The dry shampoo product according to Paragraphs B-C, wherein the hexamethyldisiloxane is present from 20% to 60%, by weight of the dry shampoo product, preferably from 25% to 55%, more preferably from 30% to 50%, and even more preferably from 35% to 45%, and alternatively from 38% to 43%.
      E. The dry shampoo product according to Paragraphs A-D, wherein the oil absorbing starch comprises rice starch, corn starch, potato starch, tapioca starch, or a combination thereof.
      F. The dry shampoo product according to Paragraphs A-E, wherein the oil absorbing starch is present at 1% to 15%, by weight of the dry shampoo product, preferably from 3% to 12%, and more preferably from 6% to 10%.
      G. The dry shampoo product according to Paragraphs A-F, wherein the propellant is free of 1,1 diflouroethane.
      H. The dry shampoo product according to Paragraphs A-H, wherein the product is formulated without water and ethanol.
      I. The dry shampoo product according to Paragraphs A-I, wherein the product is formulated without polymers and surfactants.
      J. The dry shampoo product according to Paragraphs A-J, further comprising a rheology modifier and/or a polar activator modifier or a polar activator.
      K. The dry shampoo product according to Paragraph J, wherein the rheology modifier and/or the polar activator improves the homogeneity of the oil absorbing starch suspended in the carrier compared to a dry shampoo product that does not include the rheology modifier.
      L. The dry shampoo product according to Paragraphs J-K, wherein the rheology modifier comprises a water-swellable clays; wherein the water-swellable clay comprises laponite, bentolite, montmorilonite, smectite, hectorite, or a combination thereof.
      M. The dry shampoo product according to Paragraphs J-L, wherein the polar activator comprises methanol, ethanol, acetone, propylene carbonate, water, triethyl citrate, propylene carbonate, or a combination thereof.
      N. The dry shampoo product according to Paragraphs J-M, wherein the rheology modifier and/or the polar activator are present at a combined amount 0.5% to 3% rheology modifier, by weight of the dry shampoo product, preferably from 0.75% to 2%, more preferably from 1% to 1.75%, and even more preferably from 1.25% to 1.5%.
      O. An aerosol hair care product, comprising:
a. an aerosol container comprising a pressurizable container and an actuator; and
b. the dry shampoo product according to Paragraphs A-N disposed in the pressurizable container;
   wherein the aerosol personal care product is pressurized to 45 psig to 80 psig,
   preferably from 50 psig to 75 psig, and more preferably from 55 psig to 70 psig, according to the Pressure Test Method.
      P. The aerosol hair care product according to Paragraph 0, wherein the dry shampoo composition feels dry within 10 seconds of being dispensed from the container.
      Q. A method of cleaning or refreshing hair, comprising:
a. providing the aerosol hair product according to Paragraphs O-P;
b. pressing the nozzle thereby releasing the dry shampoo product as an aerosol spray;
c. applying the dry shampoo composition to a user's hair.
   R. The method according to Paragraph Q, wherein 0.025 g to 0.2 g of oil absorbing starch is applied to the hair in one second, preferably from 0.05 g/s to 0.15 g/s, more preferably from 0.06 g/s to 0.1 g/s, and even more preferably from 0.07 g/s to 0.08 g/s according to the Delivery Rate Test Method.
   S. The method according to Paragraphs Q-R, wherein the aerosol spray comprises an average particle size (Dv50) of from 5 μm to 250 μm, preferably from 10 μm to 225 μm according to the Particle Size Distribution Test Method.
   T. Use of a carrier comprising a volatile silicone having a viscosity of 1 cst or less, preferably 0.65 cst or less, in a dry shampoo product as defined in Paragraphs A-N for providing a dry shampoo composition with 55% or less volatile organic compounds, preferably 50% or less volatile organic compounds.
   U. Use of a carrier comprising a volatile silicone having a viscosity of 1 cst or less, preferably 0.65 cst or less, in a dry shampoo product as defined in Paragraphs A-N for delivering a dry shampoo that dries in approximately 10 seconds or less, absorbs oil, and/or has low visible residue after application.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dry shampoo product, comprising:
a) a carrier comprising a volatile silicone having a viscosity of 1 cst or less;
b) an oil absorbing starch dispersed in the volatile silicone carrier;
c) a propellant;
wherein the dry shampoo product comprises 55% or less of volatile organic compounds, by weight of the dry shampoo product.

2. The dry shampoo product of claim 1, wherein the dry shampoo product comprises 50% or less of volatile organic compounds.

3. The dry shampoo product of claim 1, wherein the volatile silicone has a viscosity of 0.65 cst or less.

4. The dry shampoo product of claim 2, wherein the volatile silicone is hexamethyldisiloxane.

5. The dry shampoo product of claim 4, wherein the hexamethyldisiloxane is present at greater than 20%, by weight of the dry shampoo product.

6. The dry shampoo product of claim 1, wherein the oil absorbing starch comprises rice starch, corn starch, potato starch, tapioca starch, or a combination thereof.

7. The dry shampoo product of claim 1, wherein the oil absorbing starch is present at about 3% to about 12%, by weight of the dry shampoo product.

8. The dry shampoo product of claim 1, wherein the propellant is free of 1,1 diflouroethane.

9. The dry shampoo product of claim 1, wherein the product is formulated without water and ethanol.

10. The dry shampoo product of claim 9, wherein the product is formulated without polymers and surfactants.

11. The dry shampoo product of claim 1, further comprising a rheology modifier and/or a polar activator modifier or a polar activator.

12. The dry shampoo product of claim 11, wherein the rheology modifier and/or the polar activator improves the homogeneity of the oil absorbing starch suspended in the carrier compared to a dry shampoo product that does not include the rheology modifier.

13. The dry shampoo product of claim 11, wherein the rheology modifier comprises a water-swellable clays; wherein the water-swellable clay comprises laponite, bentolite, montmorilonite, smectite, hectorite, or a combination thereof.

14. The dry shampoo product of claim 11, wherein the polar activator comprises methanol, ethanol, acetone, propylene carbonate, water, triethyl citrate, propylene carbonate, or a combination thereof.

15. The dry shampoo product of claim 11, wherein the rheology modifier and/or the polar activator are present at a combined amount of about 0.5% to about 3%, by weight of the dry shampoo product.

16. An aerosol hair care product, comprising:
a. an aerosol container comprising a pressurizable container and an actuator; and
b. a dry shampoo product disposed in the pressurizable container, the dry shampoo product, comprising:
i. a dry shampoo composition comprising:
1. a carrier comprising hexamethyldisiloxane having a viscosity of 0.65 cst or less;
2. an oil absorbing starch dispersed in the hexamethyldisiloxane volatile silicone carrier;
ii. a propellant;
wherein the dry shampoo product comprises 55% or less of volatile organic compounds, by weight of the dry shampoo product;
wherein the aerosol personal care product is pressurized to greater than about 50 psig.

17. The aerosol hair care product of claim 15, wherein the dry shampoo composition feels dry within about 10 seconds of being dispensed from the container.

18. A method of cleaning or refreshing hair, comprising:
a. providing the aerosol hair product of claim 16;
b. pressing the nozzle thereby releasing the dry shampoo product as an aerosol spray;
c. applying the dry shampoo composition to a user's hair.

19. The method of claim 18, wherein about 0.025 g to about 0.2 g of oil absorbing starch is applied to the hair in one second.

20. The method of claim 18, wherein the aerosol spray comprises an average particle size (Dv50) of from 5 μm to about 250 μm according to the particle size distribution test method.
